(19) United States
(12) Patent Application Publication
(10) Pub. No.: US 2023/0404545 A1
(43) Pub. Date: Dec. 21, 2023

(54) SPECIMEN COLLECTION DEVICE AND METHOD

(71) Applicant: Yukon Medical, LLC, Durham, NC (US)

(72) Inventors: Edward P. Browka, Rome, NY (US); Todd M. Korogi, Durham, NC (US)

(73) Assignee: Yukon Medical, LLC, Durham, NC (US)

(21) Appl. No.: 18/035,704

(22) PCT Filed: Mar. 17, 2021
(86) PCT No.: PCT/US21/22806

§ 371 Date: May 5, 2023

Related U.S. Application Data
(63) Continuation-in-part of application No. 29/757,604, filed on Nov. 6, 2020, now Pat. No. D975270.

(51) Int. Cl. A61B 10/00

(52) U.S. Cl. CPC A61B 10/0051

(57) ABSTRACT

A specimen collection device for the collection of biological samples is provided. The specimen collection device provides for the sterile and safe collection of biological samples from patients, as well as efficient removal of the biological sample from the device into a reagent solution for analysis. In one aspect, a plurality of collection devices in combination with a single regent container or vial can be part of a kit used for the diagnosis of a group of individuals.

### TECHNICAL FIELD

This disclosure relates to a collection device for obtaining a biological specimen. The presently disclosed device can be used for collecting biological samples in the nasal cavity, nasopharynx, mid-turbinates, oropharynx, throat, cheek, ear, anus, vagina, and other bodily orifices.

### BACKGROUND

In the medical field, swabs for collecting biological specimen and other material are widely used. These swabs essentially consist of a cylindrical rod with one end of the cylindrical rod wrapped or covered in fiber or “flock.” This is known as the tip, or the head, of the swab. The tip can consist of cotton, rayon, polyurethane, or nylon fibers. These materials are generally hydrophilic and absorb the sample within or on the fibers for collection. Typically, a solid plastic applicator, which can be molded into various anatomical shapes, has glue applied to the applicator tip and then thousands of short nylon strands are sprayed (or flocked) onto the tip. Foam tipped swabs are generally considered only marginally better than fiber wrapped flocked swabs because of their low sample absorption.

After sample collection, the tip of the swab is immersed in a reagent vial that comprises fluid, such as a regent solution. Often, the fluid in the reagent vial is of small volume, as it is intended for a single swab. This small volume of fluid can get substantially absorbed by the tip of the swab and become difficult to retain for analytical purposes. Many of the point of care diagnostics coming to market do not have large fluid solutions so standard specimen collection devices like flocked swabs and foam swabs would absorb all the buffer preventing the test from being able to be completed.

Another unaddressed technical problem is the absence of cost-effective, convenient methods for diagnosing groups of people in an anonymous manner to determine if quarantine of the group is warranted based on one member of the group being positive or asymptomatic of a contagious aliment. This is particularly problematic in relation to potentially deadly viral pathogens. In one example, the viral pathogen includes SARS (Severe Acute Respiratory Syndrome), COVID (Coronavirus), or influenzas.

### SUMMARY

In one example, a specimen collection device is provided, the specimen collection device comprising a handle portion, and a tip portion coupled to the handle, the tip portion having a proximal end terminating in a distal end, where the tip portion has a surface, wherein the tip portion comprises a plurality of flutes, channels, or lands and grooves in the surface.

In another aspect, alone or in combination with any one of the previous aspects, the handle and tip portion are co-linear. In another aspect, alone or in combination with any one of the previous aspects, the tip portion is generally a truncated conic shape, a cylindrical shape, or tapered conic or cylindrical shape. In another aspect, alone or in combination with any one of the previous aspects, the at least a portion of the surface is hydrophobic, slightly hydrophilic and essentially non-absorbing, or a polymeric material with moisture absorption of less than 0.5 weight percent as measured in accordance with ISO 62 (2008).

In another aspect, alone or in combination with any one of the previous aspects, the tip portion comprises a plurality of flutes in the surface, the flutes radially extending about a circumference of the tip portion. In another aspect, alone or in combination with any one of the previous aspects, the tip portion comprises a plurality of lands and groves arranged circumferentially about the tip portion. In another aspect, alone or in combination with any one of the previous aspects, the tip portion comprises a plurality of continuous, nonlinear flutes generally parallel with a longitudinal axis of the tip portion, the plurality of continuous, nonlinear flutes defining one or more collection volumes. In another aspect, alone or in combination with any one of the previous aspects, the tip portion comprise a first surface rib extending from the proximal end to the distal end, the plurality of flutes extending from the first surface rib.

In another aspect, alone or in combination with any one of the previous aspects, at least one of the plurality of flutes terminates at a second surface rib, the second surface rib extending from the proximal end to the distal end, the second surface rib distally positioned from the first surface rib about the tip portion.

In another aspect, alone or in combination with any one of the previous aspects, at least two of the plurality of flutes extend radially about the circumference of the tip portion from the first surface rib in opposite direction terminating at the second surface rib and define at least one collection volume. In another aspect, alone or in combination with any one of the previous aspects, at least one of the plurality of flutes adjacent the proximal end is substantially parallel to a longitudinal axis of the specimen collection device. In another aspect, alone or in combination with any one of the previous aspects, at least two of the plurality of flutes or grooves vary in pitch from the proximal end to the distal end; or the lands and the grooves are discontinuous and/or vary in aspect ratio.

In another aspect, alone or in combination with any one of the previous aspects, the tip portion comprises an elastomeric material. In another aspect, alone or in combination with any one of the previous aspects, the tip portion is configured to flex in in multiple directions relative to the handle. In another aspect, alone or in combination with any one of the previous aspects, the tip portion is configured to flex in less than all directions relative to the handle.

In a second example, a method of improving sample collection from a collection device is provided, the method comprising contacting at least one of the collection device defined by any one of the previous aspects with a reagent solution, wherein each of the one or more collection devices comprises an amount of biological sample contained on the surface of the tip portion, the flutes, channels, lands and grooves, or collection volumes; and releasing the amount of biological sample from the surface of the tip portion, the flutes, lands or grooves, channels, or collection volumes into the reagent solution.

In another aspect, alone or in combination with any one of the previous aspects, at least one of the collection device comprises two or more collection devices.

In another aspect, alone or in combination with any one of the previous aspects, at least one of the collection device comprises two or more collection devices and he amount of biological sample on the surface of the tip portion, the flutes, lands or grooves, channels, or collection volumes are collected in the same reagent solution.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a perspective view of a mid-turbinate collection device as disclosed and described herein.

FIG. 1B is a perspective view of FIG. 1A rotated 90 degrees about its longitudinal axis.

FIG. 1C is a perspective view of FIG. 1B rotated 90 degrees about its longitudinal axis.

FIG. 1D is a perspective view of FIG. 1C rotated 90 degrees about its longitudinal axis.

FIG. 1E is top view of FIG. 1A.

FIG. 1F is a side view of FIG. 1A.

FIG. 1G is a side view of FIG. 1F rotated 90 degrees about its longitudinal axis.

FIG. 1H is a side view of FIG. 1G rotated 90 degrees about its longitudinal axis.

FIG. 1I is a side view of FIG. 1H rotated 90 degrees about its longitudinal axis.

FIG. 1J is an enlarged detail view of section 1J of FIG. 1F, as disclosed and described herein.

FIG. 1K is a section view of section 1J along section line 1K-1K.

FIG. 1L is a section view of section 1J along section line 1L-1L.

FIG. 1M is a section view of section 1J along section line 1M-1M.

FIG. 1N is enlarged detail view of a section 1J of FIG. 1F, as disclosed and described herein.

FIG. 2A is a perspective view of an alternate mid-turbinate collection device as disclosed and described herein.

FIG. 2B is a perspective view of FIG. 2A rotated 90 degrees about its longitudinal axis.

FIG. 2C is a perspective view of FIG. 2B rotated 90 degrees about its longitudinal axis.

FIG. 2D is a perspective view of FIG. 2C rotated 90 degrees about its longitudinal axis.

FIG. 2E is a top view of FIG. 2A.

FIG. 2F is a perspective view of FIG. 2B showing section 2G

FIG. 2G is an enlarged detailed view of section 2G.

FIG. 3A is a perspective view of a nasal collection device as disclosed and described herein.

FIG. 3B is a perspective view of FIG. 3A rotated 90 degrees about its longitudinal axis as disclosed and described herein.

FIG. 3C is a perspective view of FIG. 3B rotated 90 degrees about its longitudinal axis as disclosed and described herein.

FIG. 3D is a perspective view of FIG. 3C rotated 90 degrees about its longitudinal axis as disclosed and described herein.

FIG. 3E is a top view of FIG. 3A.

FIG. 3F is a side view of FIG. 1A.

FIG. 3G is a side view of FIG. 3F rotated 90 degrees about its longitudinal axis.

FIG. 3H is a side view of FIG. 3G rotated 90 degrees about its longitudinal axis.

FIG. 3I is a side view of FIG. 3H rotated 90 degrees about its longitudinal axis.

FIG. 4A is a perspective view of an alternative nasal collection device as disclosed and described herein.

FIG. 4B is a perspective view of the nasal collection swab of FIG. 4A rotated 90 degrees about its longitudinal axis.

FIG. 4C is a perspective view of the nasal collection swab of FIG. 4B rotated 90 degrees about its longitudinal axis.

FIG. 4D is a perspective view of the nasal collection swab of FIG. 4C rotated 90 degrees about its longitudinal axis.

FIG. 4E is a top view of the nasal collection swab of FIG. 4A.

FIG. 5A is a perspective view of an alternative nasal collection device as disclosed and described herein.

FIG. 5B is a perspective view of the nasal collection device of FIG. 5A rotated 90 degrees about its longitudinal axis as disclosed and described herein.

FIG. 5C is a perspective view of the nasal collection device of FIG. 5B rotated 90 degrees about its longitudinal axis as disclosed and described herein.

FIG. 5D is a perspective view of the nasal collection device of FIG. 5C rotated 90 degrees about its longitudinal axis as disclosed and described herein.

FIG. 5E is a top view of the nasal collection device of FIG. 5A.

FIG. 6A is a perspective view of a nasopharyngeal collection device as disclosed and described herein.

FIG. 6B is a perspective view of the nasopharyngeal collection device of FIG. 6A rotated 90 degrees about its longitudinal axis as disclosed and described herein.

FIG. 6C is a perspective view of the nasopharyngeal collection device of FIG. 6B rotated 90 degrees about its longitudinal axis as disclosed and described herein.

FIG. 6D is a perspective view of the nasopharyngeal collection device of FIG. 6C rotated 90 degrees about its longitudinal axis as disclosed and described herein.

FIG. 6E is an enlarged detail top view of the nasopharyngeal collection device of FIG. 6A.

FIG. 6F is a perspective view showing section 6G of the device of FIG. 6C, as disclosed and described herein.

FIG. 6G is an enlarged detail view of section 6F of the nasopharyngeal collection device of FIG. 6F.

FIG. 6H is a perspective view showing section 61 of the device of FIG. 6D, as disclosed and described herein.

FIG. 6I is an enlarged detail view of section 61 of the device of FIG. 6H.

FIG. 7A is a side view of an alternate nasopharyngeal collection device, as disclosed and described herein.

FIG. 7B is an enlarged detail top view of FIG. 7A as disclosed and described herein.

FIG. 7C is an enlarged detail view of a section 7C of FIG. 7A as disclosed and described herein.

FIG. 7D is a perspective view of the nasopharyngeal collection device of FIG. 7A rotated 90 degrees about its longitudinal axis as disclosed and described herein.

FIG. 7E is an enlarged detail view of a section 7E of the device of FIG. 7D, as disclosed and described herein.

FIG. 8A is sectional view of a collection volume of a collection device showing an exemplary aspect ratio, before and after specimen collection, as disclosed and described herein.

FIG. 8B is sectional view of a collection volume of a collection device showing an exemplary aspect ratio, before and after specimen collection, as disclosed and described herein.

FIG. 8C is sectional view of a collection volume of a collection device showing an exemplary aspect ratio, before and after specimen collection, as disclosed and described herein.

FIG. 9A is perspective view of a collection device showing, as disclosed and described herein.

FIG. 9B is an enlarged view of section 9B of the collection device of FIG. 9A.

FIG. 9C is sectional view of tip portion along section line 9C-9C of FIG. 9B.

FIG. 9D is perspective view of the collection device of FIG. 9A, rotated approximately 90° about its longitudinal axis.

FIG. 9E is an enlarged view section 9D of the collection device of FIG. 9D.

### DETAILED DESCRIPTION

The present disclosure is directed to a specimen collection device providing a technical solution to the above-described technical problems with conventional “flock” tipped biological specimen collection devices. The present disclosure provides a specimen collection device comprising, in one example, a hydrophobic or non-absorbent tip material. In one example, the present disclosure provides a specimen collection device comprising, in one example, a hydrophobic or non-absorbent tip portion of a hydrophobic or non-absorbent material, the surface having a plurality of flutes, or channels, or cut-outs in the surface. In at least one example, the presently disclosed collection devices are devoid of flock.

As used herein, the terms and phrases “flutes” and “collection volume” and “lands and grooves” are used interchangeably to describe a structural attribute of at least a portion of the presently disclosed device.

As used herein, the term “non-absorbing” includes its customary meaning and encompasses polymeric materials with less than 0.5 weight percent moisture absorption at equilibrium at 23° C./50% relative humidity in accordance with ISO-62 (2016). In one example, non-absorbing polymeric materials suitable for the tip portion of the presently disclosed device have less than 0.1 weight percent moisture absorption at equilibrium at 23° C./50% relative humidity in accordance with ISO-62 (2008).

In one example, the surface of the presently disclosed device is configured to obtain biological material upon the hydrophobic or non-absorbent surface of the tip portion, rather than absorbing biological material into the tip portion. In one example, biological material in contact with the hydrophobic or non-absorbent surface of the tip portion is at least partially released into the fluid of a reagent container. In one example, biological material in contact with the hydrophobic or non-absorbent surface of the tip portion is essentially completely released into the fluid of a reagent container.

In one example, the collection device is capable of collecting an effective amount of sample material. In one example, the collection device does not retain fluid when immersed in a solution. This functionality of the presently disclosed sample collection device provides for test assays that require small amounts of solution.

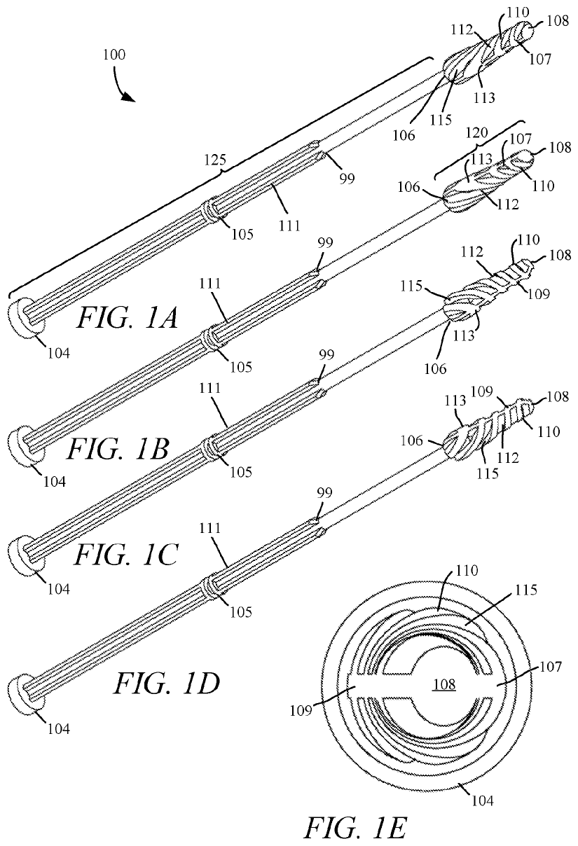

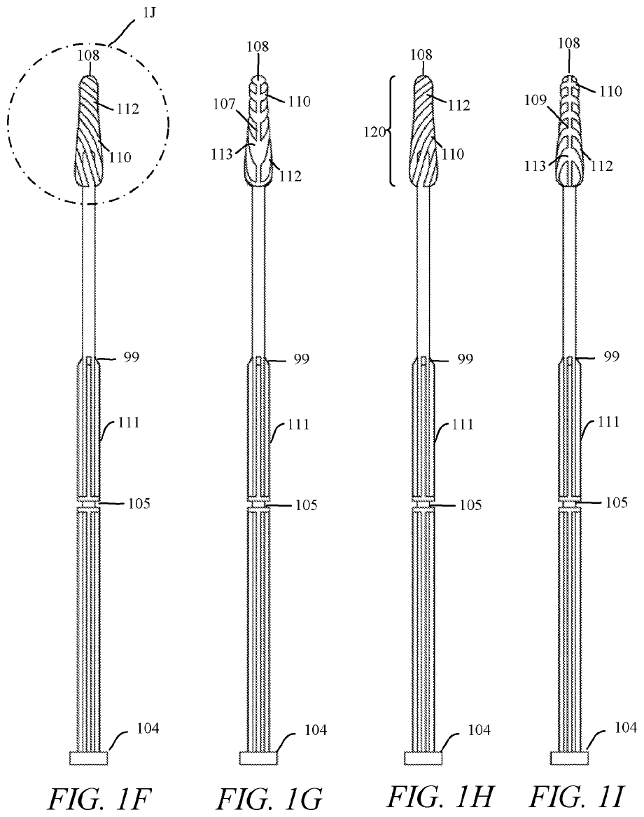

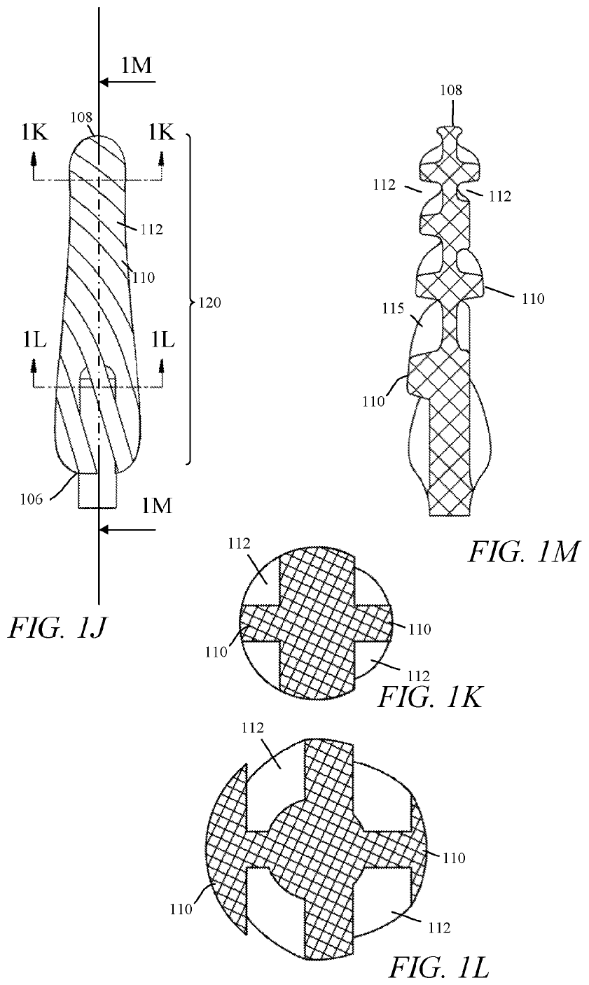

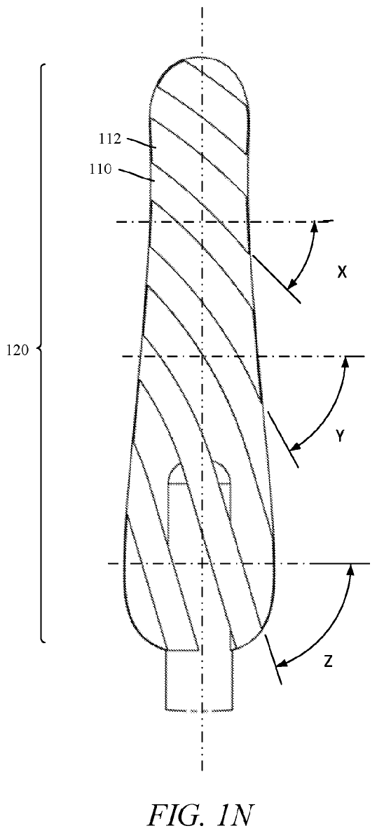

Although nasopharyngeal, mid-turbinate, and nasal devices are shown, the presently disclosed device can be used and is applicable for collecting biological samples in the nasal cavity, nasopharynx, mid-turbinates, oropharynx, throat, cheek, ear, anus, vagina, and other bodily orifices. Thus, with reference to FIGS. 1A-1D, a perspective view of an exemplary mid-turbinate collection device 100, where the collection device 100 comprises an end member 104, a handle 125 extending from the end member, the handle comprising spaced apart ribs 111 extending co-linear with the handle, the handle comprising an optional break point 105, the handle terminating at proximal end 106 of tip portion 120. In one example, the collection device 100 comprises a handle 125 coupled to a connecting member (not shown) which receives, or is coupled to, the tip portion 120.

In one example, end member 104 is configured with an outer diameter larger than the outer diameter of the tip portion 120 so as to avoid accidental insertion of the wrong end of the device 100 into a subject. In another example, end member 104 is configured with an outer diameter at least 1.5×, 2×, 3×, 4× or 5× the diameter of tip portion 120.

As shown, ribs 111 include chamfer 99 on the end of the rib that transitions into the handle. Chamfer 99 provides reduced stress concentration and/assists with break at an intended break point while preventing or eliminating break elsewhere. In one example, the ribs 111 of handle 125 are configured for facilitating spinning the device with fingers rather than the wrist.

The tip portion 120 has proximal end 106 terminating at distal end 108. In one example, tip portion 120 is generally cylindrical. In another example, tip portion 120 is generally conic with a taper from the proximal end 106. In one example, tip portion 120 is generally truncated conic with an inward taper from the proximal end 106 and an outward taper therefrom towards the distal end 108. In one example, tip portion 120 has a taper configured to aid insertion past or around one or more anatomical features present in the orifice during sample collection. In one example, tip portion 120 is configured with a “Coca-Cola®-bottle shape” like tapering (dip in/dip out) from the proximal end 106 to the distal end 108 for assisting navigation through the anatomy of the orifice and/or for increased comfort to the subject during sample collection.

In one example, tip portion 120 is constructed of a hydrophobic or nonabsorbent polymeric material. In one example, tip portion 120 includes a plurality of flutes 110 (“lands”) defining collection volumes 112 (“grooves” or spiral cuts) in tip portion surface. Flutes 110 extend mid-plane from longitudinally extending support ribs 107 and 109. The flutes 110 have edges, and are shown as crisp edges. In one example, flutes 110 have filleted or chamfered edges so as to reduce part stress and increase comfort during sample collection. In one example, the flutes 110 have filleted or chamfered edges that are rigid so as to allow for extraction and collection of more viscous or hardened mucus specimen that otherwise is not collectible using a fiber-flock swab.

In one example, flute 110 is configured with a flute width for automated specimen removal/washing. Thus, in one example, tip portion 120 is inserted into a device that flushes over the tip portion and allows for specimen collection. Flute width can be configured to maximize specimen removal as opposed to a finer flutes width that would retain more specimen when flushed by fluid coming from a single direction or small volumes of flushing fluid. The pitch of flute 110 provides improved specimen release as opposed to flutes substantially or completely orthogonal to the direction of flow (along the tip portion's longitudinal axis), while the pitch of flute 110 also provides improved specimen collection as opposed to flutes substantially or completely parallel of the tip portion's longitudinal axis.

In one example, collection device 100 is designed such that as tip portion 120 rotates, collection volume 112, as defined by flutes 110, present openings/cups to tissue/mucous and aid in collection and direct sample into the collection volume. This structural figuration is superior to flutes running strictly or substantially parallel to the tip portions longitudinal axis as there would not be an opening/cup-like collection volume presented.

FIG. 1E is top view of FIG. 1A showing mid-turbinate collection device 100, terminating in proximal end 108. In one example the proximal end 108 is flanked by support ribs 107 and 109. In one example, there are a plurality of flutes 110 extending from support ribs 107 and 109.

With reference to FIGS. 1F-1I, side views of device 100 are shown where the mid-turbinate collection device 100 comprises an end member 104, a handle 125 extending from the end member, the handle comprising spaced apart ribs 111 extending colinear with the handle, the handle comprising a break point 105, the handle terminating at connecting member 99, the connecting member 99 terminating at or within tip portion 120. The ribs 111 on the handle allow for spinning the device with fingers rather than the wrist. Tip portion 120 has a proximal end 106 terminating at distal end 108.

In one example, tip portion 120 is generally cylindrical with an inward taper from the proximal end 106 and an outward taper therefrom towards the distal end 108. As shown, tip portion 120 is has slightly wider, opposing surface lands 113 and 115 adjacent the proximal end 106 that are bisected by support ribs 107 and 109, respectively, that are configurable to maintain a constant groove width of the collection volumes 112 adjacent proximal end 106. In another example, the tip portion 120 has a surface 113 and 115 constructed of a non-absorbing hydrophilic polymeric material. In one example, the tip portion 120 has a surface 113 and 115 constructed of a non-absorbing hydrophilic polymeric material. In another example, tip portion 120 includes a plurality of flutes 110 that define a plurality of collection volumes 112.

In one example, tip portion 120 possesses mirror symmetry about a portion of its circumference, as shown in FIGS. 1F and 1H. In one example, tip portion 120 lacks mirror symmetry about a portion of its circumference, as shown in FIGS. 1G and 1I. Often times, a healthcare provider, because of fatigue or inexperience may insufficiently rotate the sample collection device during sample collection. In anticipation of this possibility, mirror symmetry about a portion of the tip portion 120 of the presently disclosed collection device provides for sample collection on one side of tip portion should only one rotational direction be used by the healthcare provider.

The flutes 110 extend approximately from one support rib 107 around the circumference and distally terminate at the other support rib 109 so as to define one or more collection volumes 112. In one example, the flutes extend in opposite directions from each support rib.

With regards to FIGS. 1J-IL, tip portion 120 comprises a proximal end 106 terminating in a distal end 108. In one example, tip portion 120 is generally cylindrical with an inward taper from the proximal end 106 and an outward taper therefrom towards the distal end 108. Tip portion comprises a plurality of flutes 110 that define a plurality of collection volumes 112. In one example, collection volumes 112 have the same volume capacity. In another example, collection volumes 112 have different volume capacities. In one example, the collection volumes 112 collects 50-120 μL.

FIG. 1M is a section view of section 1J along section line 1M-1M, wherein flutes 110 and are flanked by collection volumes 112. Tip portion 120 is generally cylindrical with a taper from the proximal end 106. The taper and width of tip portion 120 allows for maneuvering past the nasal cavity and through the mid-turbinate cartilage. In one example, the mid-plane of the tip portion 120 is flat.

FIG. 1N is an enlarged view of a section 1J of FIG. 1F, wherein the tip portion 120 comprises flutes 110 and collection volumes 112. In one example, flutes 110 extend from the latitudinal axis of the device at a pitch angle X. In one example, pitch angle Z is greater than pitch angle Y. In one example, pitch angle Y is greater than pitch angle X. In one example, pitch of flutes 110 varies. In one example, the pitch angles are configured to maximize sample collection and maximize sample release into reagent vials and/or reagent containers. In one example, each and every flute 110 can be pitched at different angles. In another example, flutes 110 can be in groups of different pitch. In another example, any other pitch arrangement can be used. For example, the pitch of a single flute 110 can be configured to change in the mid-section of the tip portion 120, whereas the pitch is more shallow at the entrance to the flute 110, and becomes progressively steeper. In one example, changing pitch of flutes 110 provides for more “scooping action” of specimen at a mouth of the collection volume 112, and allows the tail of the flute to scrape (and obtain) more specimen. Thus, the pitch of the flutes 110 of tip portion 120 are configured to provide improved sample collection by scooping and scraping as compared to a fully horizontal flute that would predominantly or only scoop, and a fully vertical flute that would predominantly or only scrap biological samples from the orifice during use.

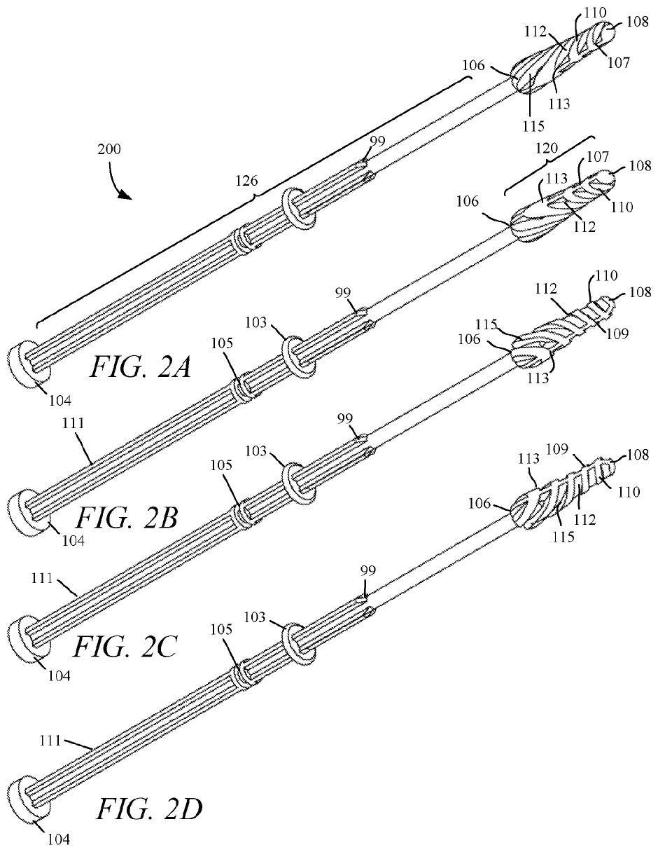

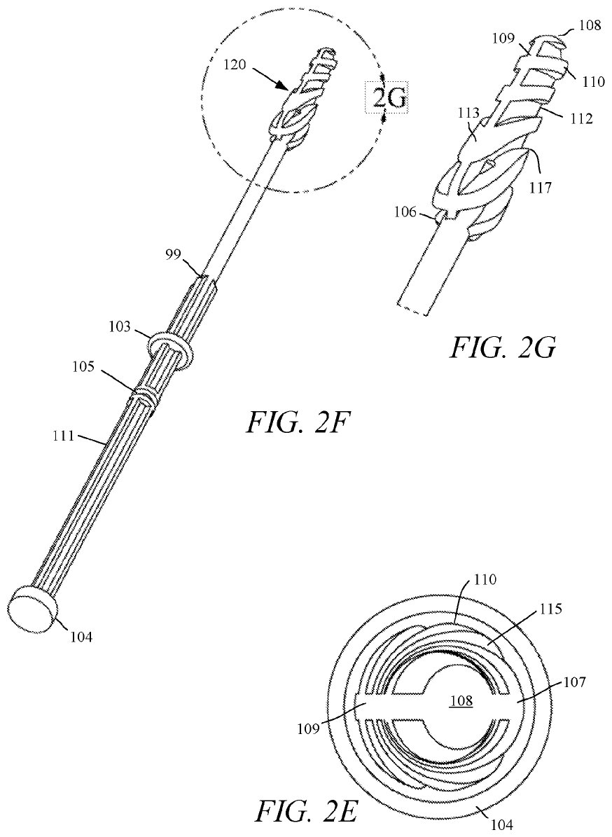

With regards to FIGS. 2A-2D, a perspective view of an exemplary alternate mid-turbinate collection device 200 is shown, where the collection device 200 comprises an end member 104, a handle 126 extending from the end member, the handle comprising spaced apart ribs 111 extending co-linear with the handle, the handle comprising a depth indicator ring 103, the handle comprising a break point 105, the handle terminating at connecting member 99, the connecting member 99 terminating at or within a tip portion 120. The ribs 111 on the handle allow for spinning the device with fingers rather than the wrist. The tip portion 120 has proximal end 106 terminating at distal end 108. In one example, tip portion 120 is generally cylindrical. In another example, tip portion 120 is generally cylindrical with a taper from the proximal end 106. In one example, tip portion 120 is generally cylindrical with an inward taper from the proximal end 106 and an outward taper therefrom towards the distal end 108. In one example, tip portion 120 is has a surface 113 and 115 constructed of a hydrophobic polymeric material. In another example, the tip portion 120 has a surface 113 and 115 constructed of a non-absorbing hydrophilic polymeric material. In one example, tip portion 120 includes a plurality of flutes 110 extending from a support rib 109 defining collection volumes 112.

FIG. 2E is a top view of FIG. 2A an alternate mid-turbinate collection device 200, terminating in proximal end 108. In one example the proximal end 108 is flanked by support ribs 107 and 109. In one example, there are a plurality of flutes 110 extending from support ribs 107 and 109. The flutes 110 define a plurality of collection volumes 112. In one example, collection volumes 112 have the same volume capacity. In another example, collection volumes 112 have different volume capacities. In one example, the collection volumes 112 collects 50-120 μL.

With reference to FIGS. 2F-2G, an enlarged detailed view of tip portion 120 is shown emphasizing plurality of flutes 110 and collection volumes 112. Edge 117 of flute 110 is shown as rounded. In one example, device 100 differs from device 200 by the presence of depth indicator ring 103 in the mid-turbinate collection device 200. The depth indicator ring 103 is absent in mid-turbinate collection device 100.

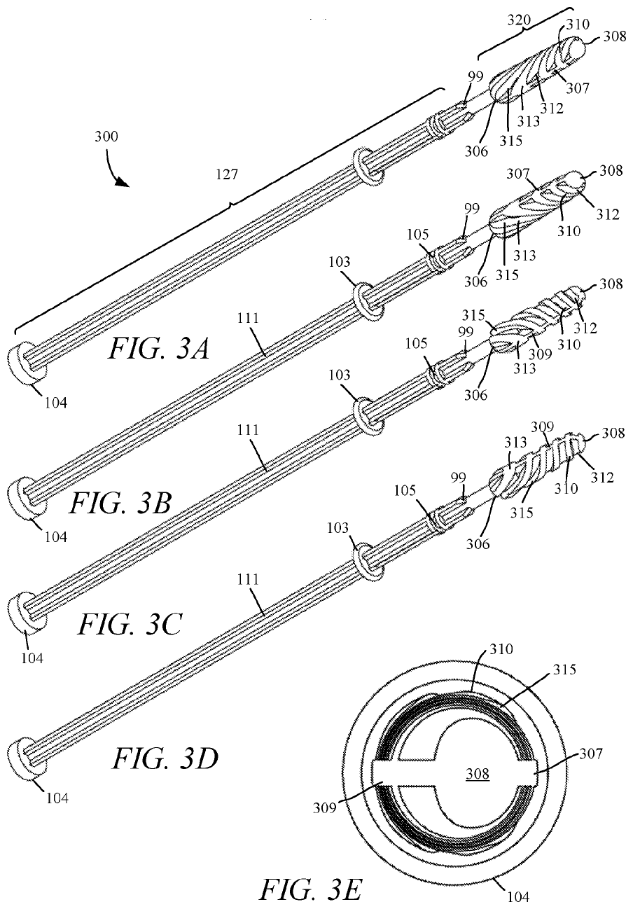

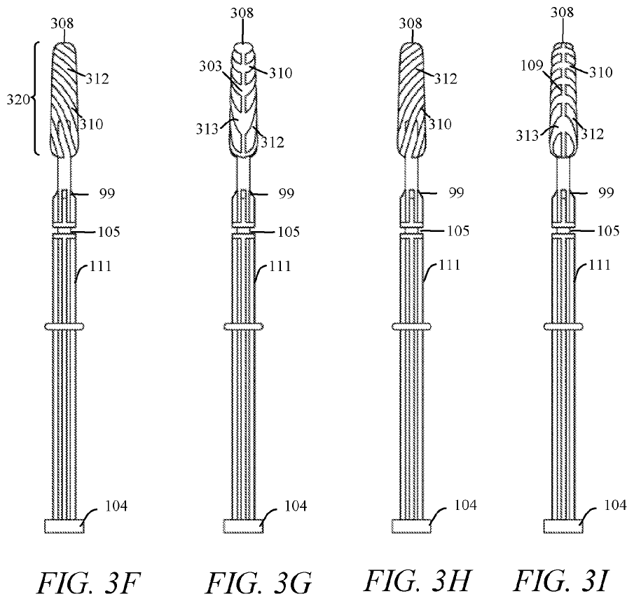

FIGS. 3A-3D are perspective views of an exemplary nasal collection device 300, where the collection device comprises an end member 104, a handle 127 extending from the end member, the handle comprising spaced apart ribs 111 extending co-linear with the handle, the handle comprising a depth indicator ring 103, the handle comprising a break point 105, the handle terminating at connecting member 99, the connecting member 99 terminating at or within a tip portion 320. The ribs 111 on the handle allow for spinning the device with fingers rather than the wrist. The tip portion 320 has proximal end 306 terminating at distal end 308. In one example, tip portion 320 is generally cylindrical. In another example, tip portion 320 is generally cylindrical with a taper from the proximal end 306. In one example, tip portion 320 is generally cylindrical with an inward taper from the proximal end 306 and an outward taper therefrom towards the distal end 108. In one example, tip portion 320 is has a surface 313 and 315 constructed of a hydrophobic polymeric material. In another example, the tip portion 320 has a surface 313 and 315 constructed of a non-absorbing hydrophilic polymeric material. In one example, tip portion 320 includes a plurality of flutes 310 extending from a support ribs 307 and 309 defining collection volumes 312. In one example, collection volumes 312 have the same volume capacity. In another example, collection volumes 312 have different volume capacities. In one example, the collection volumes 312 collects 50-120 μL.

FIG. 3E is a top view of FIG. 3A of exemplary mid-turbinate collection device 300. In one example the distal end 308 is flanked by support ribs 307 and 309. In one example, there are a plurality of flutes 310 extending from support ribs 307 and 309.

In reference to FIGS. 3F-31, a side view of mid-turbinate collection device 300 is shown where the collection device 300 comprises an end member 104, a handle 125 extending from the end member, the handle comprising spaced apart ribs 111 extending co-linear with the handle, the handle comprising a depth indicator ring 103, the handle comprising a break point 105, the handle terminating at connecting member 99, the connecting member 99 terminating at or within a tip portion 320. The ribs 111 on the handle allow for spinning the device with fingers rather than the wrist. The tip portion 320 has proximal end 306 terminating at distal end 308. In one example, tip portion 320 is generally cylindrical. In another example, tip portion 320 is generally cylindrical with a taper from the proximal end 306. In one example, tip portion 320 is generally cylindrical with an inward taper from the proximal end 306 and an outward taper therefrom towards the distal end 108. In one example, tip portion 320 is has a surface 313 and 315 constructed of a hydrophobic polymeric material. In one example, tip portion 320 includes a plurality of flutes 310 extending from a support ribs 307 and 309 defining collection volumes 312. In one example, collection volumes 312 have the same volume capacity. In another example, collection volumes 312 have different volume capacities.

In one example, tip portion 320 possesses mirror symmetry about a portion of its circumference, as shown in FIGS. 3F and 3H. In one example, tip portion 320 lacks mirror symmetry about a portion of its circumference, as shown in FIGS. 3GF and 3I. Mirror symmetry about a portion of the tip portion 320 provides for sample collection on one side of tip portion should only one rotational direction be used by the healthcare provider.

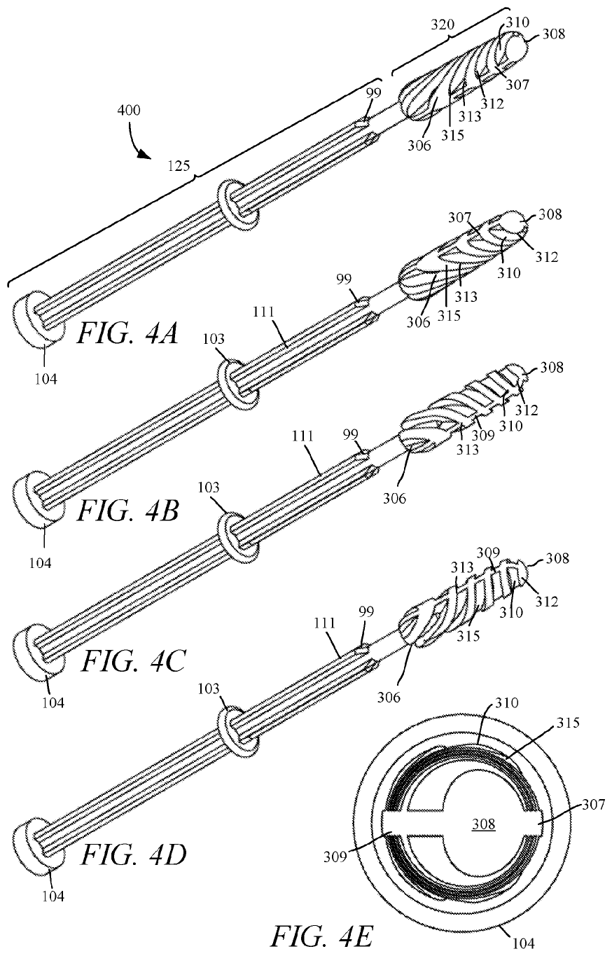

FIGS. 4A-4D shows a perspective view of an exemplary alternative nasal collection device 400, where the nasal collection device 400 comprises an end member 104, a handle 128 extending from the end member, the handle comprising spaced apart ribs 111 extending co-linear with the handle, the handle comprising a depth indicator ring 103, the handle terminating at connecting member 99, the connecting member 99 terminating at or within a tip portion 320. The ribs 111 on the handle allow for spinning the device with fingers rather than the wrist. The tip portion 320 has proximal end 306 terminating at distal end 308. In one example, tip portion 320 is generally cylindrical. In another example, tip portion 320 is generally cylindrical with a taper from the proximal end 306. In one example, tip portion 320 is generally cylindrical with an inward taper from the proximal end 306 and an outward taper therefrom towards the distal end 108. In one example, tip portion 320 is has a surface 313 and 315 constructed of a hydrophobic polymeric material. In another example, the tip portion 320 has a surface 313 and 315 constructed of a non-absorbing hydrophilic polymeric material. In one example, tip portion 320 includes a plurality of flutes 310 extending from a support ribs 307 and 309 defining collection volumes 312. In one example, collection volumes 312 have the same volume capacity. In another example, collection volumes 312 have different volume capacities. In one example, the collection volumes 312 collects 50-120 μL.

FIG. 4E is a top view of the nasal collection device 400 terminating in distal end 308 of tip portion 320. In one example the tip portion 320 is flanked by support ribs 307 and 309. In one example, there are a plurality of flutes 310 extending from support ribs 307 and 309.

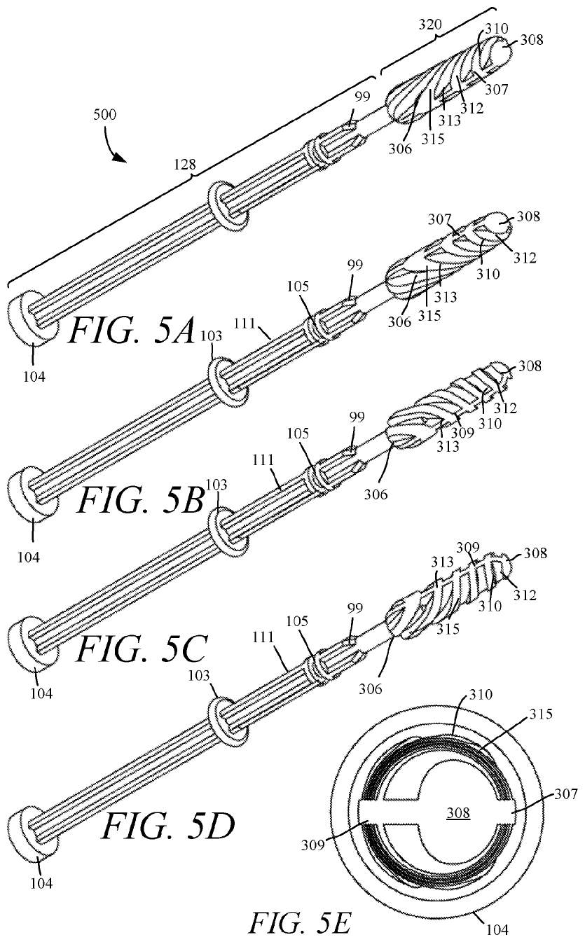

FIGS. 5A-5D is a perspective view of an exemplary alternative nasal collection device 500 where the nasal collection device 400 comprises an end member 104, a handle 129 extending from the end member, the handle comprising spaced apart ribs 111 extending co-linear with the handle, the handle comprising a depth indicator ring 103, the handle terminating at connecting member 99, the connecting member 99 terminating at or within a tip portion 320. The ribs 111 on the handle allow for spinning the device with fingers rather than the wrist. The tip portion 320 has proximal end 306 terminating at distal end 308. In one example, tip portion 320 is generally cylindrical. In another example, tip portion 320 is generally cylindrical with a taper from the proximal end 306. In one example, tip portion 320 is generally cylindrical with an inward taper from the proximal end 306 and an outward taper therefrom towards the distal end 108. In one example, tip portion 320 is has a surface 313 and 315 constructed of a hydrophobic polymeric material. In another example, the tip portion 320 has a surface 313 and 315 constructed of a non-absorbing hydrophilic polymeric material. In one example, tip portion 320 includes a plurality of flutes 310 extending from a support ribs 307 and 309 defining collection volumes 312. In one example, collection volumes 312 have the same volume capacity. In another example, collection volumes 312 have different volume capacities. In one example, the collection volumes 312 collects 5-200 μL (microliters), 10-175 μL, or 15-150 μL.

FIG. 5E is a top view of the nasal collection device 500 terminating in tip portion 320. In one example the distal end 308 is flanked by support ribs 307 and 309. In one example, there are a plurality of flutes 310 extending from support ribs 307 and 309.

In one example, nasal collection device 400 differs from nasal collection device 300 and 500 by the absence of break point 105. In one example, mid-turbinate collection devices 100, 200 differ from nasal collection devices 300, 400, 500 in size and taper of the tip portions 120 and 320 respectively. In one example, tip portion 320 of devices 300, 400, 500 is wider than tip portion 120 of devices 100, 200. In one example, tip portion 320 of devices 300, 400, 500 is more blunt than tip portion 120 of devices 100, 200 to prevent over-insertion.

As shown in FIGS. 1A-5E, tip portions 120, 320 comprises flutes 110, 310 configured with a shallow attack angle that get steeper from the distal end to the proximal end so as to allow for improved collection as well as improved flushing and release of specimen. The steepness of the flutes 110,310 (close to parallel to shaft at proximal end 106,306) and their respective collection volumes 112,312 allows for collection even if device is only twisted in one direction. The flutes 110,310 form a plurality of collection volumes 112, 312. The flutes are aligned along a support rib 307 and 309 that extends in line with the handle 125. The tip portion 120, has slight flaring to allow for easy insertion. In one example, the handle 125 comprises a frangible break point 105 for sterile specimen collection technique. In one example, the handle comprises depth indicator ring 103 to limit insertion and communicate visually whether the device can be used for nasopharyngeal, nasal, or mid-turbinate collection. The handle 125 also has ribs 111 to help grip during user twisting and smooth edges for patient comfort. The connecting member 99 connects the handle 125 to the tip portion 120,320. In one example, tip portion 120 has slight flaring (in then out, or dip in/dip out) to allow for facile insertion into an orifice. In one example, the tip portion 120, 320 is blunt to prevent over-insertion past the nasal cavity.

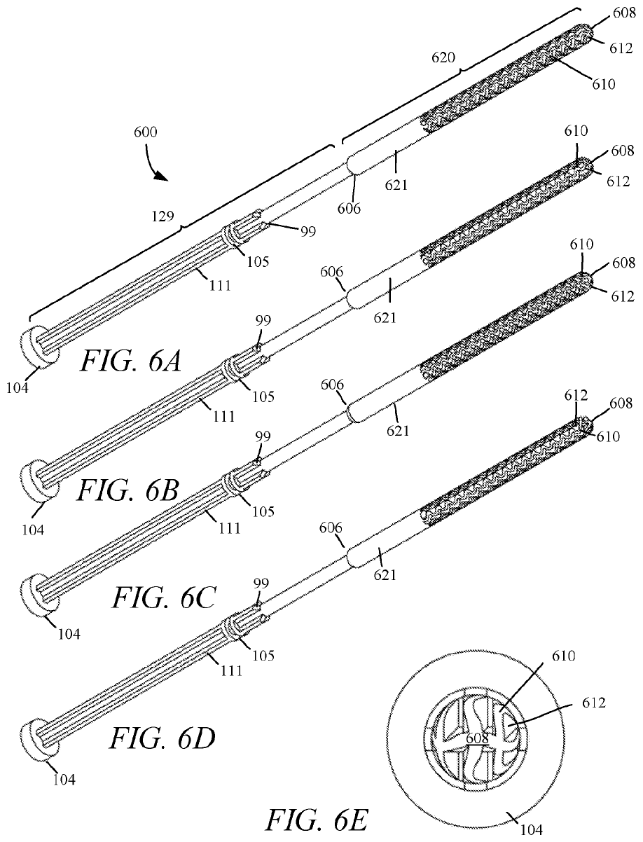

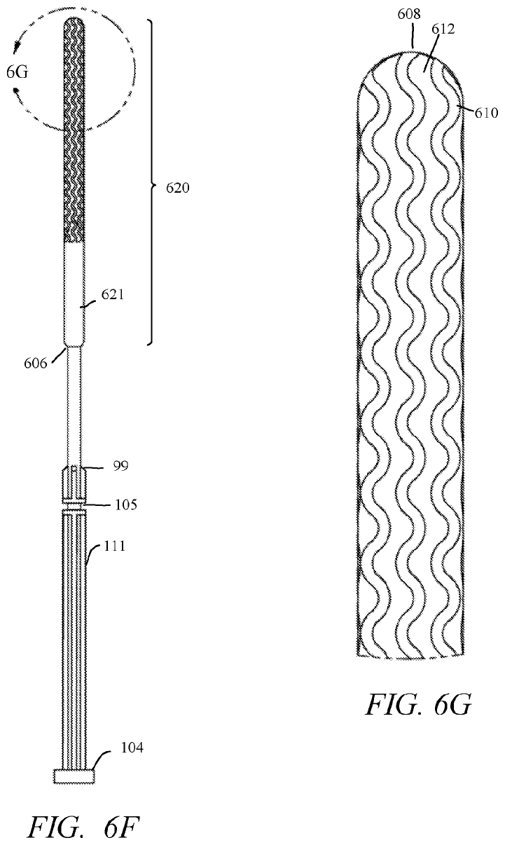

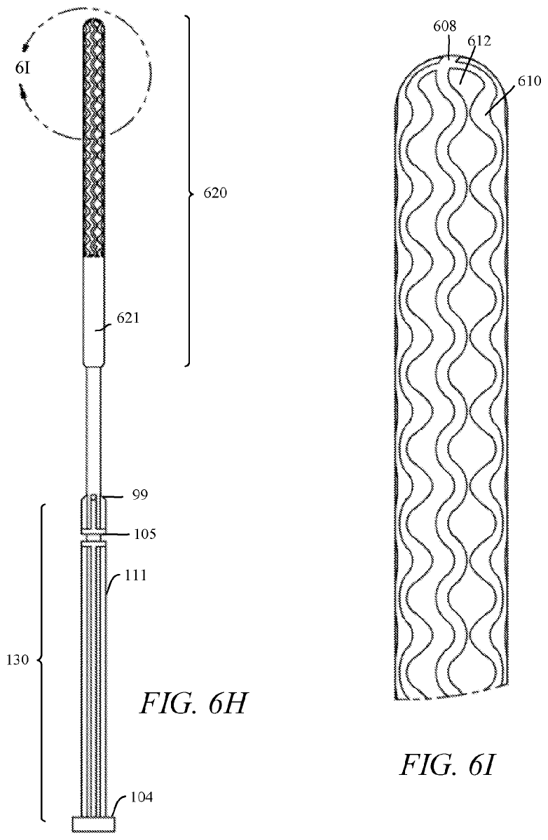

FIGS. 6A-6D shows a perspective view of an exemplary nasopharyngeal collection device 600, where the nasopharyngeal collection device 600 comprises an end member 104, a handle 130 extending from the end member, the handle comprising spaced apart ribs 111, the handle comprising a break point 105, the handle terminating or within tip portion 620. The tip portion 620 having a proximal end 606 terminating at a distal end 608. Tip portion 620 has a plurality of nonlinear lands/flutes 610 and grooves/collection volumes 612. In one example, the lands and grooves, or flutes 610 are “S-shaped.”

In one example, the tip portion 620 is generally cylindrical. In one example, at least a portion of tip portion 620 has a smooth surface portion 621. In one example, tip portion 620 is co-injected molded over the handle 125. In another example, tip portion 620 is injection molded in combination with laser milling of the S-shaped collection volumes 612. As shown, tip portion 620 has a proximal smooth portion 621 adjacent the proximal end 606, the plurality of S-shaped lands/flutes 610 extending from smooth surface portion 621 to the distal end 608. S-shaped lands/flutes 610 define a plurality of S-shaped grooves/collection volumes 612. In one example, the tip portion 620 is comprised of an elastomeric material.

FIG. 6E is an enlarged detail top view of distal end 608 of the nasopharyngeal collection device 600. In one example, the distal end 608 connects a plurality of S-shaped protrusions 610 and S-shaped collection grooves/collection volumes 612. As shown, the tip portion 620 lacks longitudinal symmetry as viewed from the top so as to provide flexibility of the tip portion in all directions during use.

FIG. 6F and FIG. 6H show opposite side views, respectively, of nasopharyngeal collection device 600. Opposite sides of collection device 600 have slightly different lands/grooves configurations. FIG. 6I is an enlarged detail view of a section 61 of the device of FIG. 6H, where S-shaped lands/flutes 610 define a plurality of nonlinear grooves/collection volumes 612. Collection volumes 612 of tip portion 620 can continuous, discontinuous or semicontinuous. As shown in FIG. 6I, grooves/collection volumes 612 are less continuous, forming a plurality of at least partially isolated volumes along the longitudinal length of tip portion 620.

Collection volume 612 formed of nonlinear lands/flutes 610 as shown is structurally different from the mid turbinate and nasal design above that have a flat mid plane tip portions. The nonlinear shaped lands/flutes 610 provide a mid-plane that allows tip portion 620 to flex in all directions. For example, an applied side load from the left to the distal end 608 causes the mid plane nonlinear flutes 610 on the right to compress and those on the left to expand, in an accordion-like manner, as well as providing flex up or down as well as in and out. In another example, nonlinear lands/flutes 610 are configured to increase the total length of the rib edge, thus improving collection volume for a given amount of end-user twist.

In one example, the tip portion 620 is comprised of a material of lower durometer than the handle 125. In another example, tip portion 620 is comprised of an elastomeric material. In one example, tip portion 620 of elastomeric material is co-injected molded over handle 125 comprised of non-elastomeric material. In another example, tip portion 620 of elastomeric material is co-injected molded over handle 125 comprised of a harder durometer material than tip portion 620.

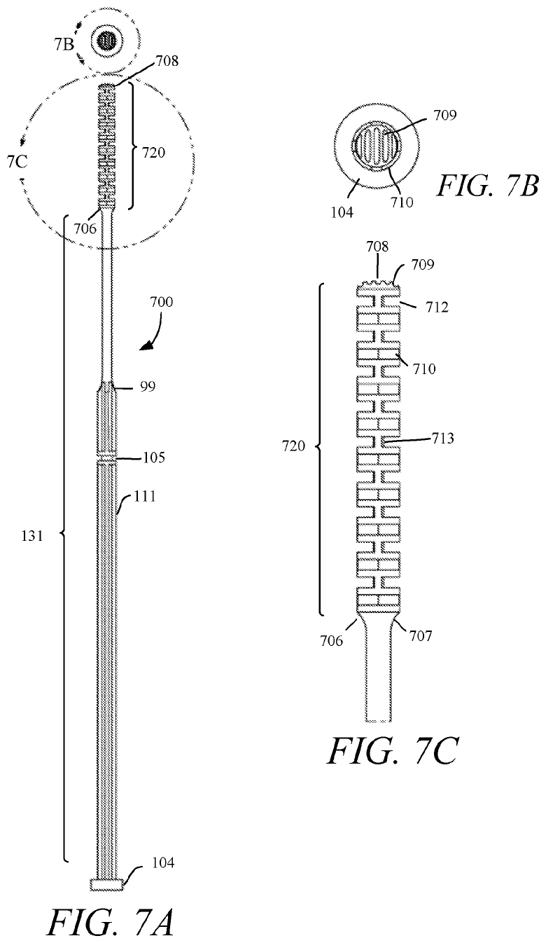

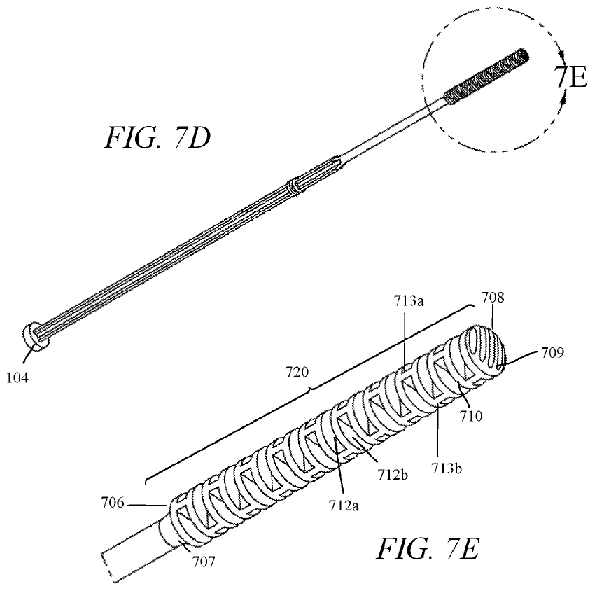

FIG. 7A is a side view of an exemplary alternate nasopharyngeal collection device 700, where the nasopharyngeal collection device 700 comprises an end member 104, a handle 131 extending from the end member, the handle comprising spaced apart ribs 111, the handle comprising a break point 105, the handle terminating at proximal end 706 of tip portion 720. Proximal end 706 terminates at distal end 708.

FIG. 7B shows an enlarged detail top view of a section 7B of FIG. 7A, where the distal end 708 comprises distal flutes 709. Distal flutes 709 create recesses between each flute, whereas these recesses provides additional collection volume. FIG. 7C is an enlarged detail view of section 7C of FIG. 7A, where the tip portion 720 comprises a plurality of longitudinally arranged discs 710 and a plurality of corresponding collection volumes 712 connected by connecting links 713. In one example, connecting links 713 are configured as mucous wipers/collectors when device 700 is rotating, and discs 710 act as wipers/collectors when inserting/withdrawing device during use. Fillet 707 at proximal end 706 as shown, eliminates or reduces stress between tip portion 720 and shaft of handle 131 which prevents or eliminates fracture at this junction. In another example, fillet 707 creates a lead out surface that reduces trauma as the tip portion 720 is withdrawn back out of the anatomy during sample collection. As every other link 713 is rotated 90 degrees, this allows for tip portion 720 to have flexibility in all directions, e.g., at least one of links 713 provides for flexing left and right, while the alternately arranged links provide for flexing up and down. Flexing can occur in combination among multiple directions e.g., down and left (parallel or perpendicular to the longitudinal axis, by all links 713 working in concert.

In one example, distal flutes 709, when device 700 is fully inserted into orifice, provide for contact with tissue at the back of the nasopharynx, which is believed to present an area of higher viral load then areas less deep in the anatomy, and thus, distal flutes 709 provide for purposely targeting collection at or in proximity to this area, and as such would increase test sensitivity/accuracy. In one example, at least a portion of edge surfaces of device 700, such as distal flutes 709 and discs 710 generally rounded so as to prevent trauma during insertion. In another example, all edge surfaces of device 700, such as distal flutes 709 and discs 710 are rounded so as to prevent trauma during insertion. The collection volumes 712 are shown perpendicular to the axis of handle 111 and its intended rotation so they can effectively wipe and collect sample as device 700 is rotated.

In one example, exemplary nasopharyngeal collection device 700, differs from exemplary nasopharyngeal collection device 600 for example, in shape and form of tip portions 620 and 720. In another example, nasopharyngeal device 600 differs from nasopharyngeal device 700 by the presence of smooth portion 621.

In one example, exemplary nasopharyngeal collection devices 600, 700 differ from exemplary mid-turbinate collection devices 100, 200 and exemplary nasal collection devices 300, 400, 500. For example, these exemplary devices can differ by the presence of or lack of depth indicator ring, spatial relationship of the depth indicator ring and/or breakpoint and/or the tip portion relative to each other, the presence or absence of a breakpoint, the tip portion shape and/or the collection volume shape. In addition, the alternating flexible links 710 of device 700 allow for flex in all directions, whereas device 100 allows for flex about its mid plain.

FIG. 7D is a perspective view of the nasopharyngeal collection device of FIG. 7A rotated 90 degrees about its longitudinal axis, and FIG. 7E is an enlarged detail view of a section 7E of the device of FIG. 7D. Disc edges 713a, 713b are shown perpendicular relative to each other, thus, providing collection volumes 712a, 712b that are presented perpendicular to each other to the targeted specimen collection area. Disc edges 713a, 713b are configured for scraping of the targeted area for retrieving specimen during use.

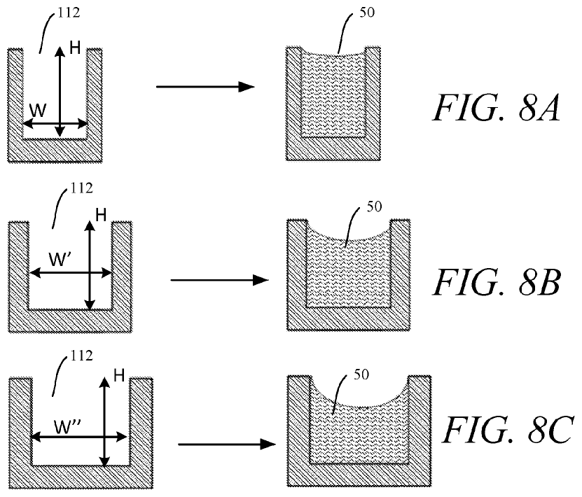

FIGS. 8A, 8B, and 8C are sectional view of collection volumes 112 of the exemplary collection device as disclosed and described herein showing various aspect ratios (Height (H)/Width (W)), before and after specimen collection. FIG. 8A depicts an aspect ratio (of the collection volume 112) of approximately 2:1 (H:W), whereas FIG. 8B depicts an intermediate aspect ratio (less than that of FIG. 8A) of approximately 1:1 (H:W′), and FIG. 8C depicts an intermediate aspect ratio (less than that of FIG. 8B) of approximately 2:3 (H:W″). The present devices provide for design of aspect ratios that are capable of providing different modes of collection, e.g., liquid specimen can be wicked in, while more viscous can be scraped in by the rib edges, or both modes are available in combination. Thus, when more viscous sample is presented to the present device, the number/total length of collection rib edges are designable to a target range of specimen per rotation. The total collection volume to be collected is related to the volume of open space to volume of plastic/elastomeric material in the tip portion. For example, a tip portion with a higher rib/land density (at the same flute/groove depth) collects more per rotation but provides a lower total collection volume than a lower rib density at the same flute/groove depth. The present devices provide tip portions configured to collect as much specimen as possible at a given tip portion total collection volume. The width of the rib/land is configured to have sufficient structure while avoiding or minimizing sharp edges. In one example, rib/land width is between about 0.25 mm to about 5 mm. In one example, rib/land heights are about 1 mm to about 2 mm radius.

With reference to FIGS. 8A., 8B, and 8C, a 2:1 aspect ratio provides approximately 45% theoretical fluid collection capability 50, while a 1:1 aspect ratio provides about 52% theoretical fluid collection capability, and a 2:3 aspect ratio provides about 42% theoretical fluid collection capability, respectively. The percentages of theoretical fluid collection volumes are estimates of actual collection volume, but are useful for tip portion design and intended end use.

In one example, a collection volume is a minimum rib/land width and maximum land height, while maintaining a tip portion diameter appropriate for the anatomical space intended use. In some conditions the target specimen for collection will form more of a meniscus, in such cases, as the aspect ratio (H/W) decreases the percent of specimen versus air in the groove also decreases.

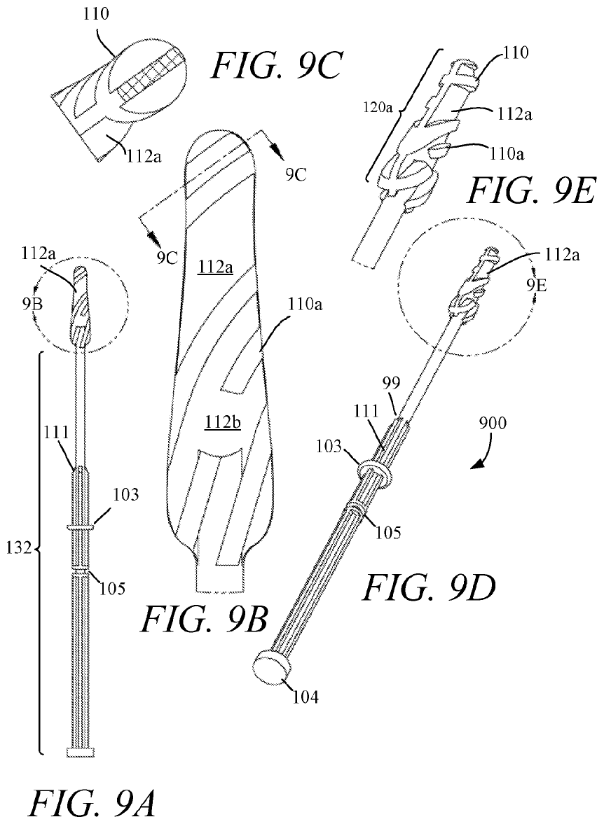

Between patients and also within patients, the viscosity of specimen varies. Thus, with reference to FIGS. 9A-9E, collection device 900 is depicted with varied groove width and non-continuous ribs/lands 110a (e.g., a portion of a rib is omitted) in tip portion 120a attached to handle 132. For example, multiple groove spacings, with varying aspect ratios in the tip portion provides for both higher ratio grooves 112b that can more fully fill with low viscosity specimen and smaller ratio grooves 112a that can more fully fill with higher viscosity, solid, and/or sticky specimen. Sectional view of tip portion 120a along section line 9C-9C of FIG. 9B, is shown in FIG. 9C, where rib 110 (or land) has variable height (front to back), e.g., it is tallest in the center and decreases to zero as it reaches the edge. Wider groove spacing provides for scraping modality of specimen collection by allowing tissues to bulge more into the grooves 112a, creating more localized force and steeper angle of attack at the scraping edge of rib 110a.

In one example, collection volume is a minimum groove width and maximum land height. In one example, the height or width of collection volume, as measured along the longitudinal axis of handle, can be 0.5-5.0 cm, 0.75-4.0 cm, 1.0-3.5 cm; 1.0-3.0 cm, or 1.0-2.5 cm, or 1.0-2 cm. in one example, the depth of collection volume, as measured perpendicular to the longitudinal axis of handle, can be 0.2-3.0 cm, 0.25-2.5 cm, 0.5-2.0 cm; 0.75-1.75 cm, or 1.0-2.5 cm, 1.0-2 cm, or 2.0-3.0 cm.

In one example, the collection volumes 112, 112a, 312, 612, 712, 712a, 712b have the same volume capacity. In another example, collection volumes 112, 312, 612, 712, 712a, 712b have different volume capacities. In one example, the collection volumes 112, 112a, 312, 612, and 712 are configured to collect at least 20 μL, at least 30 μL, at least 40 μL or more of biological sample from the subject. In one example, the collection volumes 112, 112A, 312, 612, and 712, 712a, 712b are configured to collect 50-120 μL of biological sample from the subject.

In one example, the presently disclosed collection devices 100, 200, 300, 400, 500, 600, 700, 900 are used in combination with a reagent container or reagent vial as a kit. In one example, the kit comprises one or more collection devices in combination with one reagent container or reagent vial. In one example, the kit is used to test a group of people, for example an entire classroom, a cohort of employees, a military barracks, squadron, division, or submarine, cruise ship, a family gathering, religious or entertainment event, or any group or social gathering. In one example, a plurality of collection device 100, 200, 300, 400, 500, 600, 700, 900 are immersed into one reagent vial or, rinsed, and discarded. In one example, the device tip is removed from the handle of the device and placed into the reagent vial or reagent container.

In another example, the device tip is not removed from the handle of the device, but spun or repeatedly dipped into the reagent vial or reagent container. In the above example, a single reagent vial or reagent container exposed to a plurality of the presently disclosed device 100, 200, 300, 400, 500, 600, 700, 900 used to test a group of individuals is tested to determine whether the tested group would need to be quarantined. In one example, the collection device described herein would reduce test interference and/or minimize regent material as it would not absorb all the reagent solution, culture solution, or other liquids in in the reagent container or vial.

This kit provides a solution during a pandemic, such as the COVID, where it may be desirable for anonymity of testing large groups of people and providing a quarantine protocol. The presently disclosed kit will provide infection results for a group anonymously. Therefore, it can be determined whether a group is infected, without pointing out which individual of the group introduced the infection.

In some embodiments, a collection device is inserted into a subject's upper respiratory tract to collect a specimen for analysis. The tip portion of the nasal device is placed in a reagent vial or chamber, or the tip portion is broken off into the reagent vial or chamber. The reagent vial or chamber is sealed and sent for analysis. In some embodiments, the specimen is taken via a nasopharyngeal device, mid-turbinate device, nasal device, oropharyngeal device, anterior nares device, or buccal device.

The presently disclosed devices can be manufactured using conventional polymer processing equipment, for example, via an injection molding method. In other embodiments, the devices are prepared via a co-injection molding method, via a 2-shot injection method. The handle and connecting member can be molded with a rigid material such as filled PP (polypropylene) or HDPE (high-density polyethylene). The tip can be molded with a similar material or a softer material such as TPE (thermoplastic elastomer) or TPU (thermoplastic polyurethane) elastomer. In other embodiments, the handle, connecting member, and tip are molded with the same material. In other embodiments, the handle, and connecting member are molded with the same material and the tip portion is molded with a different material (e.g., softer). In some embodiments, the tip portion flutes and/or collection volumes are laser milled or formed with lithography methods from a solid tip portion. The collection devices 100, 200, 300, 400, 500, 600, 700, 900 can be manufactured and aseptically packaged and/or sterilized using conventional methods such as e-beam, ethylene oxide, gamma radiation.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

1. A biological specimen collection device comprising:
handle portion; and
a tip portion, the tip portion having a proximal end coupled to the handle portion, the proximal end terminating in a distal end;
wherein the tip portion has a surface, wherein the tip portion comprises a plurality of flutes, channels, or lands and grooves in the surface.

2. (canceled)

3. The biological specimen collection device of claim 1, wherein the tip portion is generally conical shaped, generally cylindrical, generally tapered conical, or generally tapered cylindrical.

4. The biological specimen collection device of claim 1, wherein the at least a portion of the surface is hydrophobic, slightly hydrophilic and essentially non-absorbing, or a polymeric material with moisture absorption of less than 0.5 weight percent as measured in accordance with ISO 62 (2008).

5. The biological specimen collection device of claim 1, wherein the tip portion comprises a plurality of lands and groves arranged circumferentially about the tip portion.

6. The biological specimen collection device of claim 1, wherein the tip portion comprises a first surface rib extending from the proximal end to the distal end, the plurality of flutes extending circumferentially from the first surface rib.

7. The biological specimen collection device of claim 1, wherein at least one of the plurality of flutes terminates at a second surface rib, the second surface rib extending from the proximal end to the distal end, the second surface rib distally positioned from the first surface rib about the circumference of the tip portion.

8. The biological specimen collection device of claim 1, wherein at least two of the plurality of flutes extend radially about the circumference of the tip portion from the first surface rib in opposite directions terminating at the second surface rib and define at least one collection volume.

9. The biological specimen collection device of claim 1, wherein: at least two of the plurality of flutes or grooves vary in pitch from the proximal end to the distal end; or the lands and the grooves are discontinuous and/or vary in aspect ratio.

10. The biological specimen collection device of claim 1, wherein at least two of the plurality of flutes decrease in pitch from the proximal end to the distal end.

11. The biological specimen collection device of claim 1, wherein at least one of the plurality of flutes adjacent the proximal end is substantially parallel to the handle.

12. The biological specimen collection device of claim 1, wherein the tip portion comprises a plurality of isolated channels in the surface, the channels radially extending about a circumference of the tip portion.

13. The biological specimen collection device of claim 1, wherein the tip portion comprises a plurality of continuous, nonlinear flutes generally parallel with a longitudinal axis of the tip portion, the plurality of continuous, nonlinear flutes defining one or more collection volumes.

14. The biological specimen collection device of claim 1, wherein the handle comprises a plurality of ribs, the plurality of ribs terminating in a chamfer, the chamfer distal from the tip portion.

15. The biological specimen collection device of claim 1, further comprising a breakpoint and/or a stop member, the breakpoint and/or the stop member positioned distally from the proximal end of the tip portion.

16. The biological specimen collection device of claim 15, wherein the breakpoint is more distal from the proximal end of the tip portion than the chamfer.

17. The biological specimen collection device of claim 15, wherein the breakpoint is more distal from the proximal end of the tip portion than the stop member.

18. The biological specimen collection device of claim 15, wherein the breakpoint is less distal from the proximal end of the tip portion than the stop member.

19. The biological specimen collection device of claim 1, wherein the tip portion comprises an elastomeric material.

20. (canceled)

21. (canceled)

22. A kit comprising:
at least one of a collection device comprising:
a handle portion; and
a tip portion, the tip portion having a proximal end coupled to the handle portion, the proximal end terminating in a distal end;
wherein the tip portion has a surface, wherein the tip portion comprises a plurality of flutes, channels, or lands and grooves in the surface; and
a reagent solution.

23. (canceled)

24. (canceled)
